(19) United States
(12) Patent Application Publication
(10) Pub. No.: US 2009/0246864 A1
(43) Pub. Date: Oct. 1, 2009

(54) Cell culture device and methods for manufacturing and using the cell culture device

(76) Inventor: Paul M. Szlosek, Kennebunk, ME (US)

Correspondence Address: CORNING INCORPORATED, SP-TI-3-1, CORNING, NY, 14831, US

(21) Appl. No.: 12/075,836

(22) Filed: Mar. 14, 2008

(51) Int. Cl. C12M 1/00; B29C 39/00

(52) U.S. Cl. 435/297.5; 264/259; 264/478

(57) ABSTRACT

A cell culture device, a method for manufacturing the cell culture device using an In Mold Labeling (IML) technique, and a method for using the cell culture device are all described herein. The cell culture device is an In Line Molded frame which has a cell growth film permanently bonded thereto. The cell growth film can be a film coated with, for example, three-dimensional randomly oriented electrospun polyamide nanofibers, a hydrogel formulation, (meth)acrylate monomers or polymers, urethane (meth)acrylate monomers or polymers, or epoxide formulation.

### TECHNICAL FIELD

The present invention relates in general to the cellular biological field and, in particular, to a cell culture device, a method for manufacturing the cell culture device, and a method for using the cell culture device.

### BACKGROUND

Manufacturers of cell culture devices have been trying to come-up with better ways of attaching a cell growth material to a cell culture device to enable the growth of cells on top of the cell growth material. In the past, the manufacturer would die cut the cell growth material and insert it into a cell culture device (e.g., Petri dish, microplate, flask, multi-layered flask). This procedure presented issues with orientation and also allowed the cell cultivating media (which contains the cells) to get below the cell growth material, neither of which is desirable. Plus, this procedure limited the ability to use microscopy to observe the growth of the cells due to the fact that the cell growth material would often have a non-planar surface. Accordingly, there has been and is a need to address this shortcoming and other shortcomings associated with the traditional cell culture device. This need and other needs have been addressed by the present invention.

### SUMMARY

In one aspect, the present invention includes a method for manufacturing a cell culture device by using a molding device and an In Line Mold Labeling technique to permanently bond a cell growth film to a moldable material to form the cell culture device. The cell culture device can be a wide variety of devices including, for example, a Petri dish, a microplate, a flask and a multi-layered flask. Plus, the cell growth film can be a film coated with, for example, three-dimensional randomly oriented electrospun polyamide nanofibers, a hydrogel formulation, urethane acrylate monomers, or an epoxide formulation.

In another aspect, the present invention includes a cell culture device with an In Line Molded frame which has a cell growth film permanently bonded thereto. The cell culture device can be a wide variety of devices including, for example, a Petri dish, a microplate, a flask and a multi-layered flask. Plus, the cell growth film can be a film coated with, for example, three-dimensional randomly oriented electrospun polyamide nanofibers, a hydrogel formulation, urethane acrylate monomers, or an epoxide formulation.

In yet another aspect, the present invention includes a method for manufacturing a cell culture device where the method includes the steps of: (a) cutting a cell growth film into a predetermined shape; (b) applying the cell growth film to a loading fixture; (c) inducing a static charge to the cell growth film which was applied to the loading fixture; (d) attaching a cell surface of the cell growth film which has a static charge to a portion of a core which is part of a molding device; (e) removing the loading fixture from the cell growth film such that the cell growth film remains attached to the core; (f) placing a die over at least the portion of the core which has the cell growth film attached thereto, where the die is also part of the molding device; (g) injecting a material within a space between the die and a bare surface of the cell growth film that was applied to the core and a space between the die and another portion of the core that does not have the cell growth film applied thereto; (h) cooling the injected material within the molding device; (i) moving the die away from the core which has the cooled material attached thereto; and (j) ejecting the ejected material from the core, where the cooled material is the cell culture device which has the cell growth film permanently bonded thereto.

In still yet another aspect, the present invention includes a method for using a cell culture device where the method includes the steps of: (a) sterilizing the cell culture device which includes an In Line Molded frame with a cell growth film permanently bonded thereto; (b) applying cells to a surface of the cell growth film within the cell culture device; and (c) allowing the cells to grow on the surface of the cell growth film within the cell culture device.

Additional aspects of the invention will be set forth, in part, in the detailed description, figures and any claims which follow, and in part will be derived from the detailed description, or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

A more complete understanding of the present invention may be had by reference to the following detailed description when taken in conjunction with the accompanying drawings wherein:

FIG. 1 is a perspective view of a cell culture device that has the shape of a Petri dish which was made by using an In Mold Labeling (IML) technique in accordance with the present invention;

FIGS. 2 and 3 respectively illustrate two photos of a cell growth film (which has three-dimensional randomly oriented electrospun polyamide nanofibers located thereon) that where taken before and after being bonded to the cell culture device using the IML molding technique in accordance with the present invention;

FIG. 4 is a flowchart illustrating the steps of a preferred method for manufacturing a cell culture device using the IML molding technique in accordance with the present invention;

FIGS. 5A-5I illustrates different views of the cell culture device at different steps in the manufacturing method shown in FIG. 4 in accordance with the present invention;

FIG. 6 is a perspective view of a cell culture device that has the shape of a microplate which was made by using the IML molding technique in accordance with the present invention;

FIG. 7 is a perspective view of a cell culture device that has the shape of a flask which was made by using the IML molding technique in accordance with the present invention;

FIGS. 8A-8B are diagrams of a cell culture device that has the shape of a multi-layered flask which was made by using the IML molding technique in accordance with the present invention; and

FIG. 9 is a flowchart illustrating the steps of a preferred method for using the cell culture device in accordance with the present invention.

### DETAILED DESCRIPTION

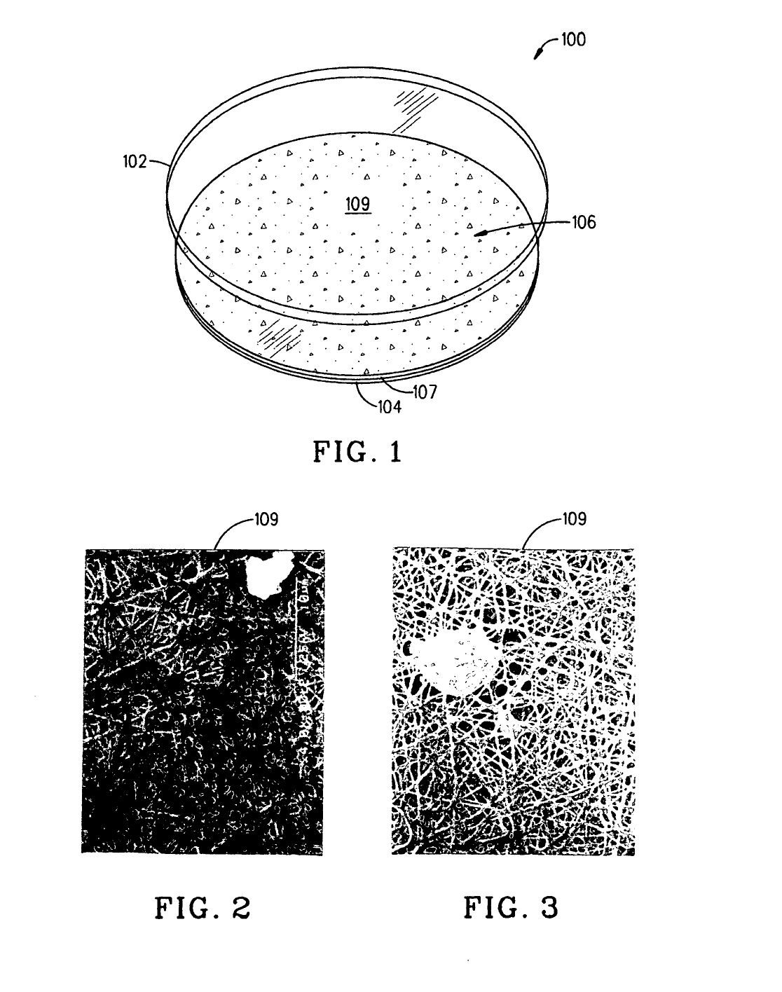

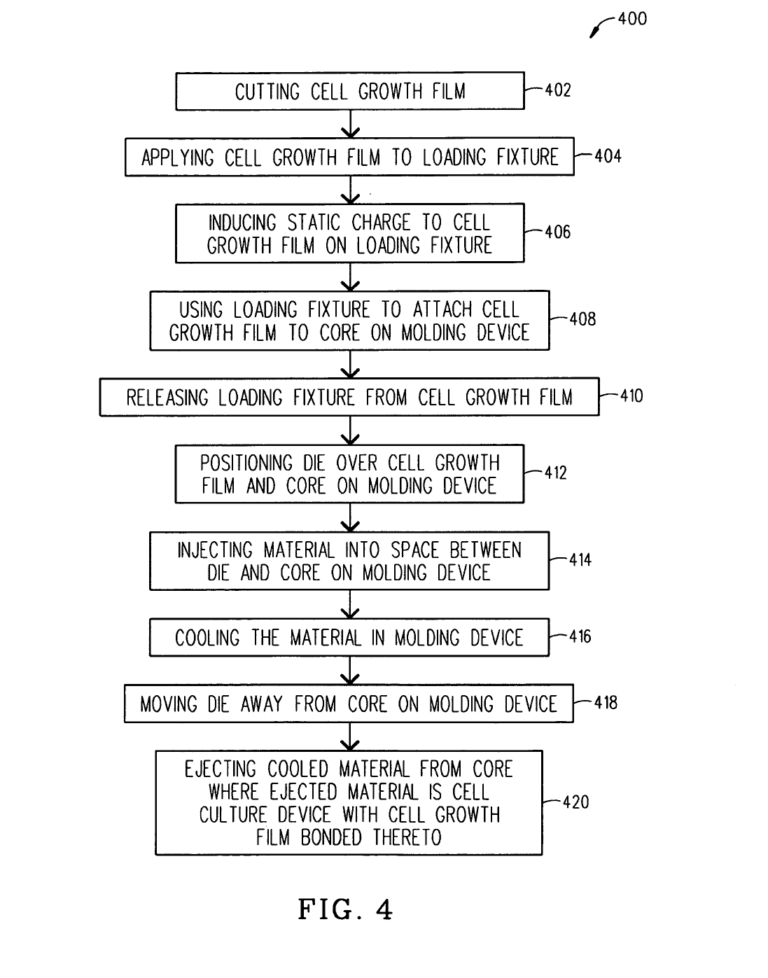

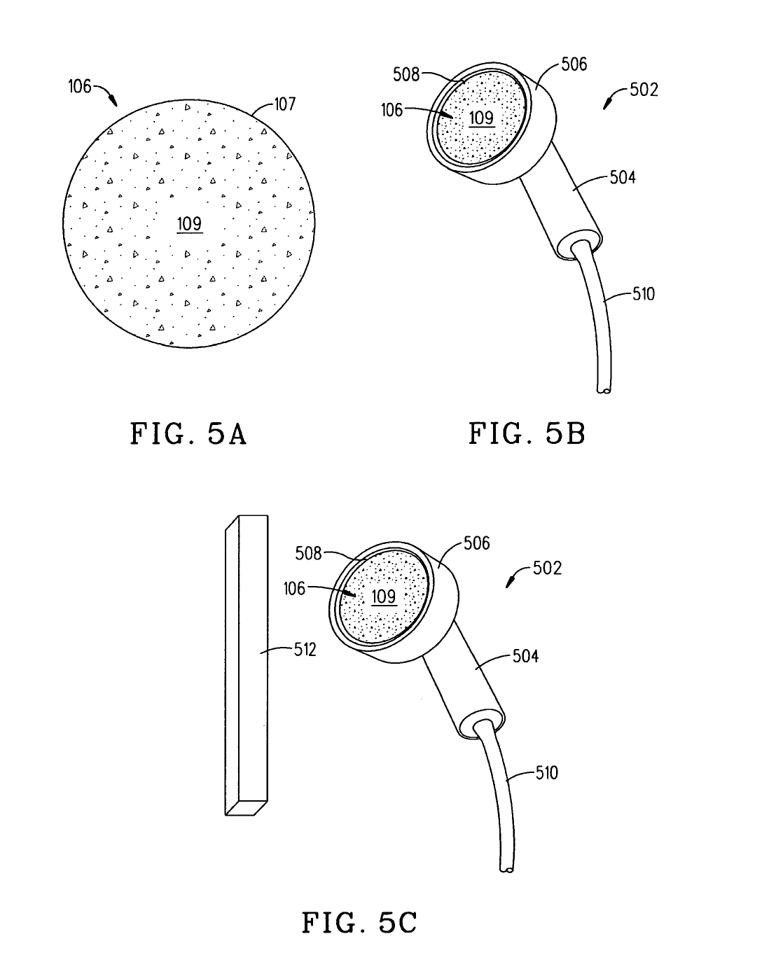

Referring to FIG. 1, there is illustrated a perspective view of an exemplary cell culture device 100 in accordance with the present invention. The cell culture device 100 includes a molded frame 102 which in this example is in the shape of a Petri dish that has a bottom surface 104 with a cell growth film 106 which was bonded thereto while molding the frame 102 using an In Mold Labeling (IML) technique. In one embodiment, the cell growth film 106 is a 0.010″ to 0.005″ thick flouropolymer film 107 (e.g., Honeywell's ACLAR film) that is coated with three-dimensional randomly oriented electrospun polyamide nanofibers 109 (see FIGS. 2 and 3 and 5E-5I). The nanofibers 109 have a fiber size distribution between 200 nm and 400 nm with an average fiber diameter of 280 nm. The nanofibers 109 create a culturing substrate that mimics a basement membrane or extracellular matrix. Thus, the nanofibers 109 offer cells a more in vitro-like fibrillar topography that, unlike biological coatings, are more stable, more consistent, and animal component-free. FIGS. 2 and 3 respectively illustrate two photos of the nanofibers 109 one taken before and one taken after the IML molding technique was used to make the cell culture device 100.

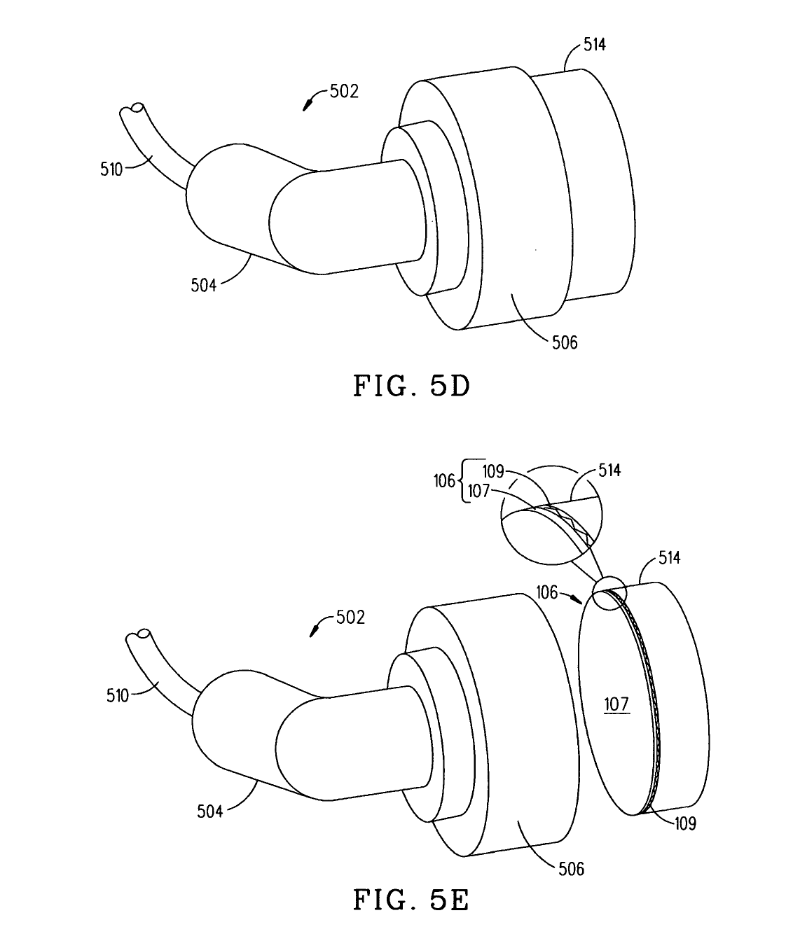

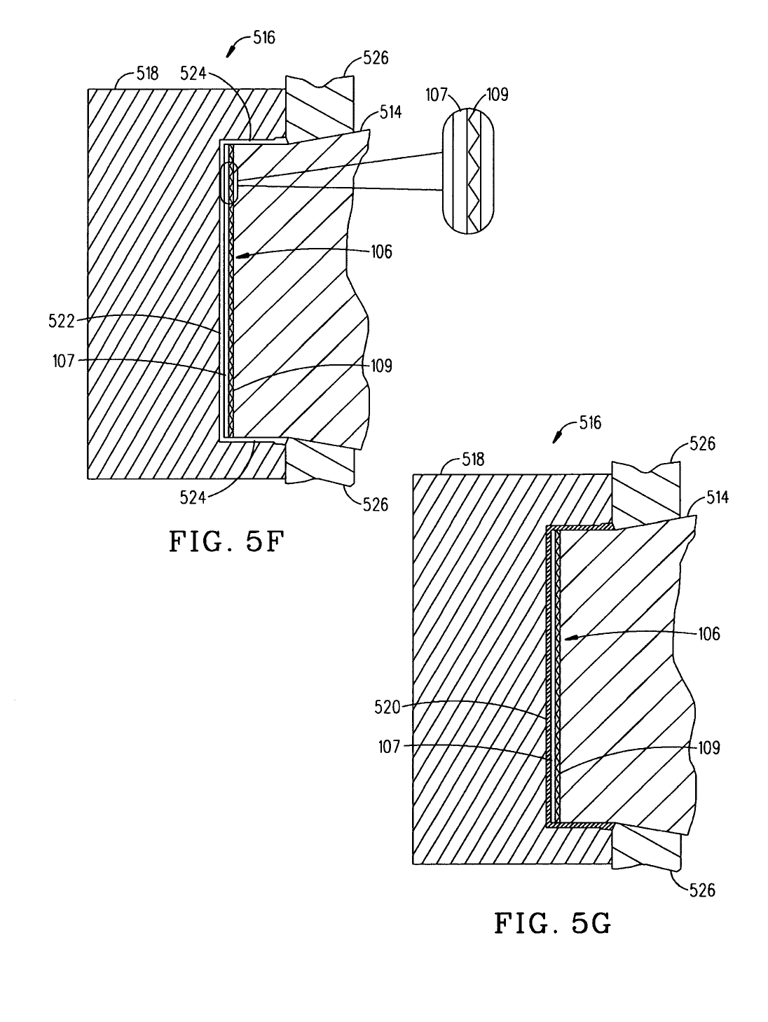

Referring to FIGS. 4 and 5A-5I, there are respectively illustrated a flowchart of a method 400 for manufacturing the cell culture device 100 and different views of the cell culture device 100 at the different steps in the manufacturing method 400. Beginning at step 402, the cell growth film 106 is cut into a predetermined shape which matches the growth area of the future cell culture device 100 (see FIG. 5A). In one embodiment, the cell growth film 106 can be rolled-up on a roll and then un-rolled before being cut into the predetermined shape desired for the future cell culture device 100. Alternatively, the cell growth film 106 can be in sheet form before being cut into the predetermined shape desired for the future cell culture device 100. The cell growth film 106 can be a film 107 that is coated with the three-dimensional randomly oriented electrospun polyamide nanofibers 109. Or, the cell growth film 106 can be a film 107 this is coated with other cell growing surfaces 109 such as, for example, a hydrogel formulation, (meth)acrylate monomers or polymers, urethane (meth)acrylate monomers or polymers, or epoxide formulation.

At step 404, the cell growth film 106 is applied to a loading fixture 502 (see FIG. 5B). In particular, the coated surface 109 (e.g., three-dimensional randomly oriented electrospun polyamide nanofibers) of the cell growth film 106 would be exposed when the cell growth film 106 is placed on the loading fixture 502. In one embodiment, the loading fixture 502 has a handle 504 attached to an application head 506 which has a depression 508 therein where the cell growth film 106 is placed and then held by a vacuum created by drawing air into the application head 506 and through the handle 504 using an air hose 510 and an external air pump (not shown).

At step 406, a static charge is induced onto the cell growth film 106 which is being held the loading fixture 502 (see FIG. 5C). In one embodiment, the loading fixture 502 holding the cell growth film 106 is passed over a static bar 512 which induces an electrical charge (e.g., negative electrical charge) onto the cell growth film 106. The purpose of inducing an electrical charge onto the cell growth film 106 will be discussed in the next step in the manufacturing process.

At step 408, the loading fixture 502 is used to attach the cell growth film 106 to a portion of a core 514 which is part of a molding device 516 (see FIG. 5D) (note: the entire molding device 516 is first shown in FIG. 5F). The electrical charge on the cell growth film 106 permits the attachment of the cell growth film 106 to the core 514 of the molding device 516. The cell growth film 106 and in particular the base film 107 needs to be able to hold the electrical charge (static charge) long enough to keep it in place on the core 514 until completion of the subsequent molding steps 410-418. In this example, the core 514 is shaped to form the inside part of the cell culture device 100 (Petri dish 100).

At step 410, the loading fixture 502 is removed from the cell growth film 106 such that the cell growth film 106 remains attached to the core 514 of the molding device 516 (see FIG. 5E). In particular, the coated surface 109 (e.g., three-dimensional randomly oriented electrospun polyamide nanofibers 109) of the cell growth film 106 would be attached to the core 514 of the molding device 516 and a bare surface 107 of the cell growth film 106 would be exposed. In one embodiment, the air flow creating the vacuum would be stopped such that loading fixture 502 would release the cell growth film 106 which would remain attached to the core 514 on the molding device 516.

At step 412, a die 518 is moved or otherwise positioned over at least the portion of the core 514 which has the cell growth film 106 attached thereto (see FIG. 5F). The die 518 is a component or part of the molding device 516 (note: the molding device 516 also has a stripper 526 which is discussed later with respect to FIG. 5I). In one embodiment, there is a space 522 between the die 518 and the bare surface 107 of the cell growth film 106 attached to the core 514 and a space 524 between the die 518 and another portion of the core 514 that does not have the cell growth film 106 attached thereto.

At step 414, a material 520 is injected within the space 522 between the die 518 and the bare surface 107 of the cell growth film 106 attached to the core 514 and a space 524 between the die 518 and another portion of the core 514 that does not have the cell growth film 106 attached thereto (see FIG. 5G). The material 520 is heated to a temperature sufficient to liquify the material 520 such that it can flow within the spaces 522 and 524 of the molding device 516. For example, the material 520 can include poly(methyl methacrylate) (PMMA), cyclic olefin copolymer (COC) (product name Topas), cyclo-olefin polymer (COP) (product name, Zeonor), styrene, polycarbonate and acrylonitrile butadiene styrene (ABS). The selection of material 520 and the film 106 has to be such that they are compatible with one another to enable a suitable bond there between.

At steps 416 and 418, the injected material 520 is allowed to cool while within the molding device 516 and then the die 518 is moved away from the core 514 which has the cooled material 520 and the cell growth film 106 attached thereto (see FIG. 5H).

At step 420, the cooled material 520 and the cell growth material 106 which has been permanently bonded thereto is ejected from the core 514 of the molding device 516. As shown, the bare surface 107 of the cell growth film 106 is the side that is permanently bonded to the cooled material 520. The ejected material 520 with the permanently bonded cell growth material 106 is the cell culture device 100 (see FIG. 5I). In one embodiment, the molding device 516 has a stripper 526 which can be moved along the core 514 to eject the cell culture device 100 from the core 514.

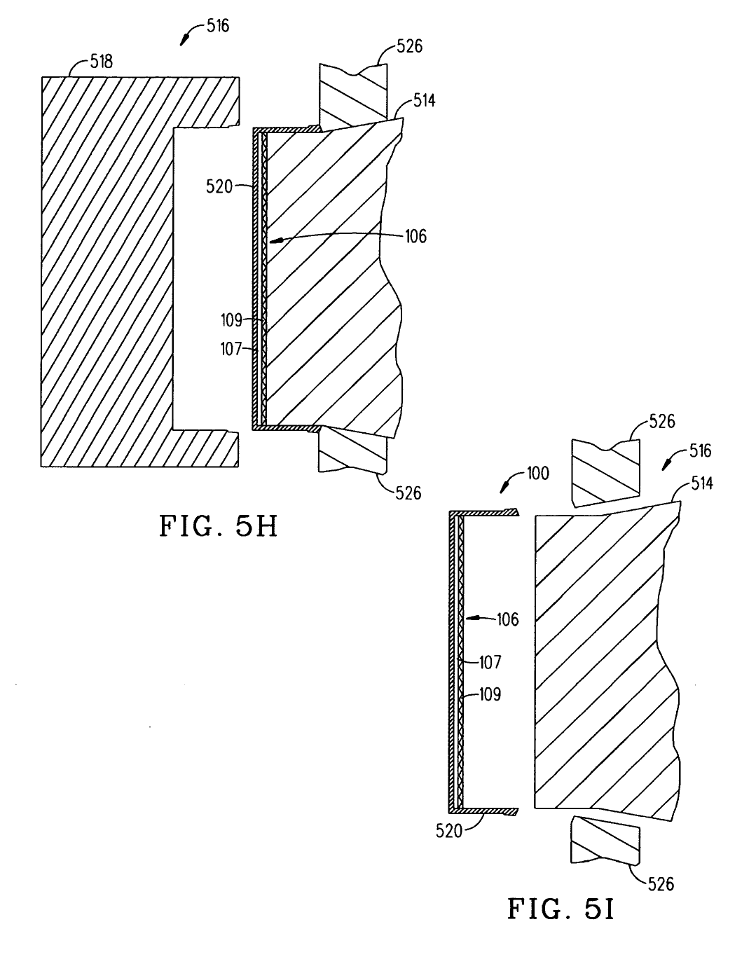

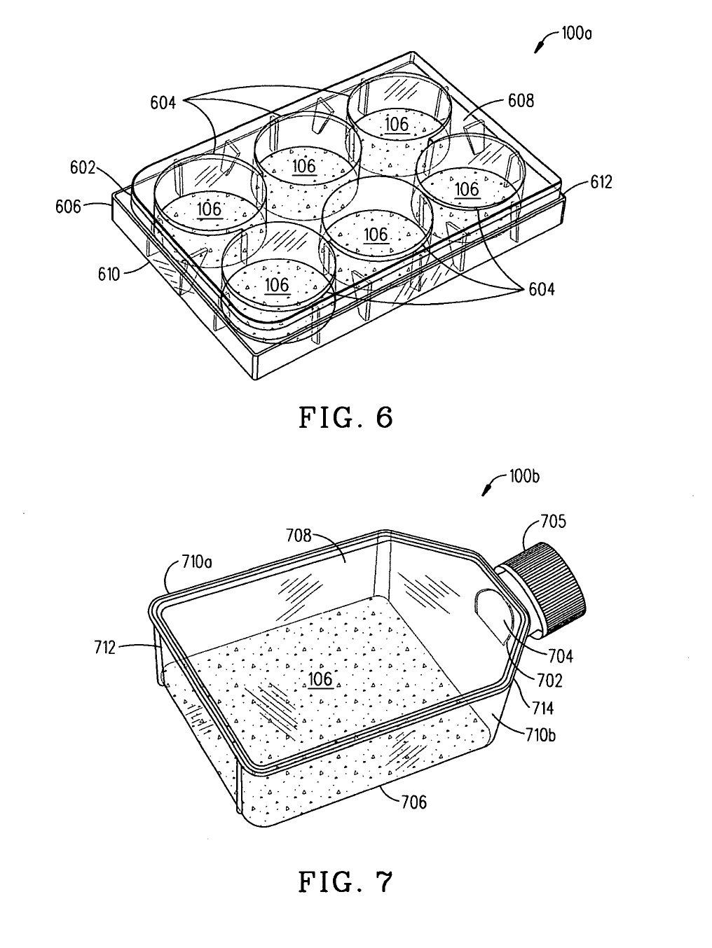

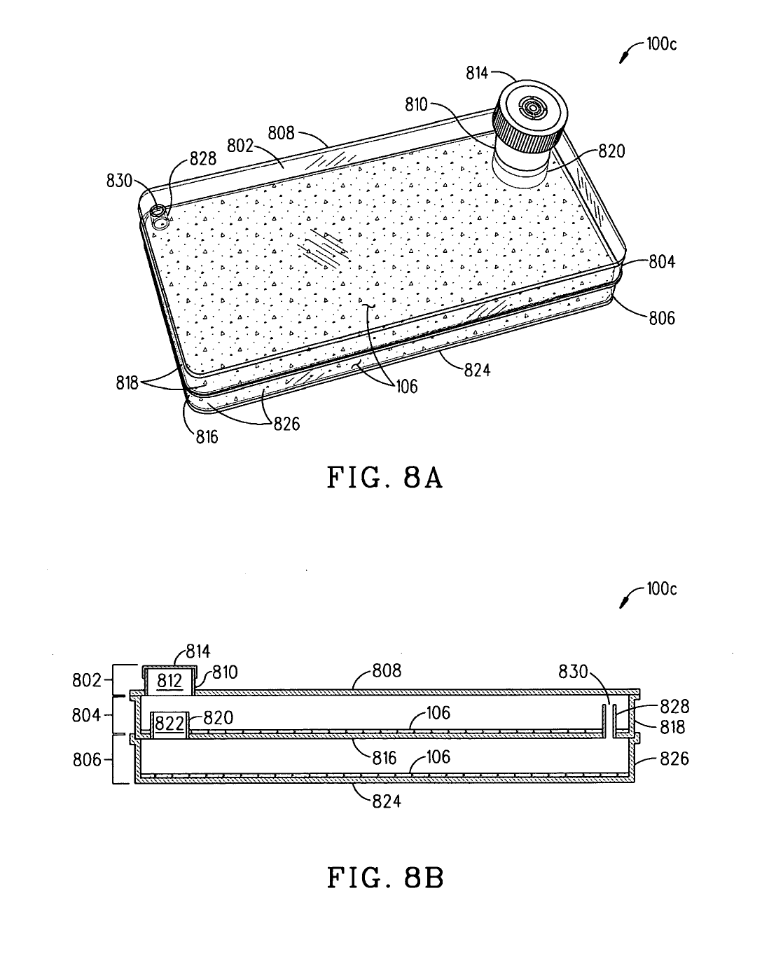

The cell culture device 100 that was manufactured using the aforementioned method 400 had the shape of a Petri dish. However, the manufacturing method 400 can be used to make different types of cell culture devices 100 such as, for example, a microplate 100a (see FIG. 6), a flask 100b (see FIG. 7) and a multi-layered flask 100c (see FIGS. 8A-8B). A detailed description about each of these devices 100a, 100b and 100c is provided below with respect to FIGS. 6-8.

Referring to FIG. 6, there is a perspective view of a cell culture device 100a that has the shape of a microplate 100a which was made by using the IML molding technique in accordance with the present invention. The exemplary microplate 100a includes a frame 602 that supports the wells 604 the bottoms of which have the cell growth film 106 which is permanently bonded thereto during the IML molding process. The frame 602 which is rectangular in shape includes an outer wall 606 and a top planar surface 608 extending between the outer wall 606 and the wells 604. However, it should be understood that the frame 602 can be provided in any number of other geometrical shapes (e.g., triangular or square) depending on the desired arrangement of the wells 604. As illustrated, the outer wall 606 that defines the outer periphery of the frame 602 has a bottom edge 610 that extends below the wells 604. Thus, when the microplate 10a is placed on a support surface, it is supported by the bottom edge 610 with the wells 604 being raised above the support surface to protect them from damage. The outer wall 606 also has a rim 612 to accommodate the skirt of a microplate cover (not shown). Although, the microplate 10a shown has six wells 604 it should be appreciated that the microplate 10a can have any number of wells 604 such as, for example, 24-wells, 96-wells and 384-wells.

Referring to FIG. 7, there is a perspective view of a cell culture device 100b that has the shape of a flask 100b which was made by using the IML molding technique in accordance with the present invention. The exemplary flask 100b was made from a transparent material but it could have also been made from a non-transparent material. The flask 100b has a neck 702 defining a filling opening 704. In this example, the neck 702 is formed with outer screw threads (not shown) for cooperating with inner screw threads (not shown) of a screw cap 705 by means of which the filling opening 704 may be closed. The flask 100b also has a flat bottom wall 706, a top wall 708, opposite side walls 710a and 710b, a flat end wall 712, and an opposite end wall 714 on which the neck 702 is formed. The bottom wall 706 has the cell growth film 106 which was permanently bonded thereto during the IML molding process.

Referring to FIGS. 8A-8B, there are respectively illustrated a perspective view and cross-sectional side view of a cell culture device 100c that has the shape of a multi-layered flask 100c which was made by using the IML molding technique in accordance with the present invention. The exemplary multi-layered flask 100c was made from a transparent material but it could also be made from a non-transparent material. In this example, the multi-layered flask 100c includes a cover 802, an intermediate tray 804 and a bottom tray 806. The intermediate tray 804 is positioned between the cover 802 and the bottom tray 806. The cover 802 includes a top plate 808 having a neck 810 that defines an opening 812 which is located near a corner of the top plate 808. The neck 810 could also have outer screw threads (not shown) for cooperating with inner screw threads (not shown) of a cap 814.

The cover 802 is attached (e.g., glued, welded, snap-fitted) to the intermediate tray 804 which has a bottom plate 816 and side walls 818 that define a cell growth area. The bottom plate 824 has the cell growth film 106 which was permanently bonded thereto during the IML molding process. The intermediate tray 804 also includes a neck 820 that defines an opening 822 which is located below the opening 812 in the cover 802. The diameter of the neck 820 in the intermediate tray 804 is smaller than the diameter of the neck 810 in the cover 802. The smaller neck 820 on the intermediate tray 804 enables a user to use a pipette (e.g., needle, syringe, capillary or similar device) to add or remove cells and cell cultivating media to or from the cell growth film 106 on the intermediate tray 804.

The intermediate tray 804 is attached (e.g., glued, welded, snap-fitted) to the bottom tray 806 which includes a bottom plate 824 and side walls 826 that define a cell growth area. The bottom plate 824 has the cell growth film 106 that was permanently bonded thereto during the IML molding process. Like with the intermediate tray 804, the user can use the pipette (or a similar device) to add or remove the cells and cell cultivating media to or from the cell growth film 106 on the bottom tray 806. As shown, the intermediate tray 804 also includes an exchange tube 828 that defines an opening 830 which is located in an opposite corner of the neck 820. The exchange tube 828 which extends up from the bottom plate 806 functions to help an operator to evenly distribute the cells and cell cultivating media between the intermediate layer 804 and the bottom layer 806 by orientating the multi-layered flask 100c in different positions.

Although the multi-layered flask 100c is described above as having one intermediate tray 804 and the bottom tray 806 on which cells can be grown, it should be understood that the multi-layered flask 100c could have any number of intermediate trays 804 and the bottom tray 806 on which to grow cells. For a detailed discussion about the structure and function of an exemplary multi-layered flask 100c without the bonded cell growth film 106 reference is made to co-assigned U.S. Pat. No. 6,569,675 entitled “Cell Cultivating Flask and Method for Using the Cell Cultivating Flask”.

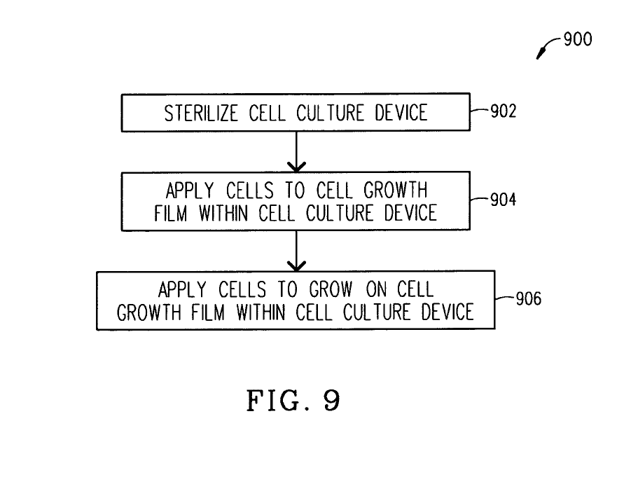

Referring to FIG. 9, there is a flowchart illustrating the steps of a preferred method 900 for using the cell culture device 100, 100a, 100b and 100c in accordance with the present invention. Beginning at step 902, the cell culture device 100, 100a, 100b and 100c is sterilized, for example, by gamma irradiation to have a sterility assurance level of 106. At step 904, the cells (which are located in a cell cultivating media) are applied to an exposed surface 109 of the cell growth film 106 within the cell culture device 100, 100a, 100b and 100c. At step 906, the cells are allowed to grow on the surface 109 of the cell growth film 106 within the cell culture device 100, 100a, 100b and 100c. Following are some optional steps that can be performed assuming the cell growth film 106 is a flouropolymer film 107 coated with the three-dimensional randomly oriented electrospun polyamide nanofibers 109.

Prior to applying the cells, the three-dimensional randomly oriented electrospun polyamide nanofibers 109 which have a slightly hydrophilic surface can be coated with a polyamine material which provides the nanofibers 109 with free amine groups for a net positive change. This treatment step may be performed because some cells prefer a positively charged surface for cell attachment. This also enables researchers to attach biomolecules to the nanofibers 109. For example, the surface modification can be achieved by covalently attaching cytokines, laminin, fibronectin, or collagen, to the polyamine-coated nanofibers 109. This step also enables one to specifically build a more in vivo-like matrix for desired cellular responses.

The cells can be fixed to the surface of the three-dimensional randomly oriented electrospun polyamide nanofibers 109 within the cell culture device 100. For example, the cells can be fixed with 4 to 85% paraformaldehyde onto the three-dimensional randomly oriented electrospun polyamide nanofibers 109. Plus, the cells may be stained for cell surface or cytochemical markers on the surface of the three-dimensional randomly oriented electrospun polyamide nanofibers 109 within the cell culture device 100, 100a, 100b and 100c.

The cells can be imaged once they are applied to the three-dimensional randomly oriented electrospun polyamide nanofibers 109 within the cell culture device 100, 100a, 100b and 100c. For instance, light microscopy, including phase contrast and differential interference contrast (DIC), can be used to view cells seeded on the cell culture device 100, 100a, 100b and 100c. The three-dimensional randomly oriented electrospun polyamide nanofibers 109 do not interfere with the imaging of the cells via fluorescence microscopy and this has been tested successfully with Texas Red, Cy3, Cy5, FITC, and GFP.

The cells can be subcultured once they have grown on the three-dimensional randomly oriented electrospun polyamide nanofibers 109 within the cell culture device 100, 100a, 100b and 100c. For instance, the cells may be subcultured using various cell dissociation techniques with trypsin, collagenase, or other enzymatic and nonenzymatic dissociation solutions. To aid with cell detachment gentle pipetting or mechanical agitation by tapping the cell culture device 100, 100a, 100b and 100c can be used. Plus, physical scrapping can be used to detach the cells.

Cells that could be applied to and grown on the three-dimensional randomly oriented electrospun polyamide nanofibers 109 within the cell culture device 100, 100a, 100b and 100c include (but are not limited to): HepG2, THLE, C3A, MDBK, MCF7, HEK293, 3T3, MRC5, BAEC, BCAEC, LNCaP, MDCK, HUVEC, PC12, Ng108, HMVEC, primary rat hepatocytes, primary rat aoritc smooth muscle, primary human chondrocytes, primary rat endothelium, primary rat astrocytes, primary rate neuronal cells, mouse embryonic stem cells, human embryonic stem cells, mesenchymal stem cells, and cord blood stem cells.

From the foregoing, it can be readily appreciated that the present invention relates to a cell culture device and a method for manufacturing the cell culture device using a decorating technique called In Mold Labeling (IML) where a film with a nano-fiber or other surface is permanently bonded to the bottom surface(s) of the cell culture device. Using the IML molding technique the film coated with nano-fibers (or other surfaces) can be in sheet or roll form and cut into to the desired shape to match the growth area of the cell culture device. It is particularly advantageous if the coated film is in roll form for continuous processes and cost considerations in the manufacturing process. The cut coated film is then given a static charge that permits it to be placed and held on the core of the IML molding device which is used to form a two dimensional growth area on the cell culture device. The molding device is closed and a melted polymer (or other material) is injected into the molding device to form the cell culture device which has the coated film permanently molded thereto. The static charge holds the coated film in place during the molding process. When the cell culture device is removed from the molding device the coated film is located in the growth area. This method works well for large surface areas that require the film to maintain a surface that is relatively flat for microscopy. It is conceivable that any surface finish or growth surface that can be applied to a film substrate could be bonded onto a cell culture surface in this manner.

The inventors have also experimented with several other alternative methods that could be used to make a cell culture device. In one alternative method, an adhesive could be used to attach the cell growth film to a previously molded cell culture product. In another alternative method, the cell growth film could be laser welded to a previously molded cell culture device. In yet another alternative method, a pressure sensitive adhesive could be used to attach the cell growth film to a previously molded cell culture device.

Although one embodiment of the present invention has been illustrated in the accompanying Drawings and described in the foregoing Detailed Description, it should be understood that the invention is not limited to the embodiment disclosed, but is capable of numerous rearrangements, modifications and substitutions without departing from the spirit of the invention as set forth and defined by the following claims.

1. A method for manufacturing a cell culture device, said method comprising the step of:
using a molding device and an In Line Mold labeling technique to permanently bond a cell growth film to a moldable material so as to form the cell culture device.

2. The method of claim 1, wherein said cell growth film is a film coated with three-dimensional randomly oriented electrospun polyamide nanofibers.

3. The method of claim 1, wherein said cell growth film is a film coated with a hydrogel formulation, (meth)acrylate monomers or polymers, urethane (meth)acrylate monomers or polymers, or epoxide formulation.

4. A cell culture device comprising:
an In Line Molded frame which has a cell growth film permanently bonded thereto.

5. The cell culture device of claim 4, wherein said cell growth film is a film coated with three-dimensional randomly oriented electrospun polyamide nanofibers.

6. The cell culture device of claim 4, wherein said cell growth film is a film coated with a hydrogel formulation, (meth)acrylate monomers or polymers, urethane (meth)acrylate monomers or polymers, or epoxide formulation.

7. A method for manufacturing a cell culture device, said method comprising the steps of:
cutting a cell growth film into a predetermined shape;
applying the cell growth film to a loading fixture;
inducing a static charge to the cell growth film which was applied to the loading fixture;
attaching a cell surface of the cell growth film which has a static charge to a portion of a core which is part of a molding device;
removing the loading fixture from the cell growth film such that the cell growth film remains attached to the core;
placing a die over at least the portion of the core which has the cell growth film attached thereto, where the die is also part of the molding device;
injecting a material within a space between the die and a bare surface of the cell growth film that was applied to the core and a space between the die and another portion of the core that does not have the cell growth film applied thereto;
cooling the injected material within the molding device;
moving the die away from the core which has the cooled material attached thereto; and
ejecting the cooled material from the core, where the ejected material is the cell culture device which has the cell growth film permanently bonded thereto.

8. The method of claim 7, further comprising a step of unrolling cell growth film from a roll before cutting the cell growth film.

9. The method of claim 7, wherein said applying step further includes a step of creating a vacuum on the loading fixture to hold the cell growth film to the loading fixture.

10. The method of claim 7, wherein said removing step further includes a step of stopping a vacuum on the loading fixture to separate the loading fixture from the cell growth film which remains attached to the core.

11. The method of claim 7, wherein said cell growth film is a film coated with three-dimensional randomly oriented electrospun polyamide nanofibers.

12. The method of claim 7, wherein said cell growth film is a film coated with a hydrogel formulation, (meth)acrylate monomers or polymers, urethane (meth)acrylate monomers or polymers, or epoxide formulation.

13. The method of claim 7, wherein said cell culture device is a petri dish, a microplate, a flask or a multi-layered flask.

14. A method for using a cell culture device, said method comprising the steps of:
sterilizing the cell culture device which includes an In Line Molded frame with a cell growth film permanently bonded thereto;
applying cells to a surface of the cell growth film within the cell culture device; and
allowing the cells to grow on the surface of the cell growth film within the cell culture device.

15. The method of claim 14, further comprising the step of imaging at least a portion of the cells on the surface of the cell growth film within the cell culture device.

16. The method of claim 14, further comprising the step of treating the cell growth film to create a net positive charge on the surface of the cell growth film within the cell culture device.

17. The method of claim 14, further comprising the step of fixing the cells on the surface of the cell growth film within the cell culture device.

18. The method of claim 14, further comprising the step of staining the cells on the surface of the cell growth film within the cell culture device.

19. The method of claim 14, further comprising the step of subculturing the cells on the surface of the cell growth film within the cell culture device.

20. The method of claim 14, wherein the cell growth film is a film coated with three-dimensional randomly oriented electrospun polyamide nanofibers.

21. The method of claim 14, wherein said cell growth film is a film coated with a hydrogel formulation, (meth)acrylate monomers or polymers, urethane (meth)acrylate monomers or polymers, or epoxide formulation.

22. The method of claim 14, wherein said cell culture device is a petri dish, a microplate, a flask or a multi-layered flask.
